(19) United States
(12) Patent Application Publication
(10) Pub. No.: US 2024/0000688 A1
(43) Pub. Date: Jan. 4, 2024

(54) COMPOSITION FOR ELIMINATING ODOR

(71) Applicant: Thnktnk LLC (Delaware), Red Bank, NJ (US)

(72) Inventor: Raymond E. Guerry, Brick, NJ (US)

(21) Appl. No.: 18/345,877

(22) Filed: Jun. 30, 2023

Related U.S. Application Data
(60) Provisional application No. 63/357,113, filed on Jun. 30, 2022.

(51) Int. Cl. A61K 8/49; A61K 8/73; A61Q 15/00; A61Q 19/10

(52) U.S. Cl. CPC A61K 8/494; A61K 8/733; A61Q 15/00; A61Q 19/10; A61K 2800/596; A61K 2800/5424

(57) ABSTRACT

A composition of odor control that includes soya-ethyl morpholinium ethosulfate (SME) and sodium alginate (ALG). The composition has a synergetic effect in eliminating residual odors. The composition can be used in a variety of formulations for handwash, laundry, spray, floor, and the like.

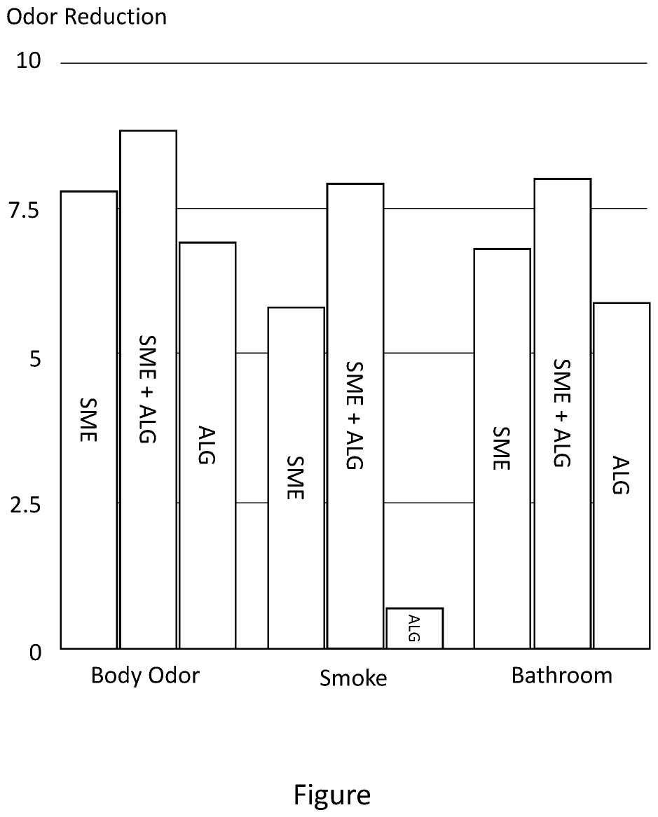

### CROSS-REFERENCE TO RELATED APPLICATIONS

This application claims priority from a U.S. Provisional Patent Application Ser. No. 63/357,113, filed on Jun. 30, 2022, the disclosure of which is incorporated herein by reference in their entirety.

### FIELD OF INVENTION

The present invention relates to a handwash composition, and more particularly, the present invention relates to a composition for eliminating odor.

### BACKGROUND

Keeping the hands clean is of the most importance for personal hygiene and to prevent the spread of contamination. Hands come in contact with different kinds of contaminants in day-to-day life. Most of the daily activities or tasks, including personal and professional, are performed using hands and in the process, the hands get contaminated. It is advised to clean the hands after most of such activities or tasks, for example, hands are washed using a soap-based composition. The soap-based compositions, with or without disinfectants, are widely used for hand cleaning. The soap-based compositions help to remove the deposited contaminants and also disinfect the hands. The soap-based compositions may also include suitable perfuming agents for deodorizing and olfactory appeal.

However, the hands often get in touch with certain substances, such as formaldehyde that leave a pungent odor on the hands that remains for a long duration event after washing the hands. Such odor from the hands is often undesirable, and thus a need is appreciated for hand cleaning composition that can eliminate pungent odors from hands.

### SUMMARY OF THE INVENTION

The following presents a simplified summary of one or more embodiments of the present invention to provide a basic understanding of such embodiments. This summary is not an extensive overview of all contemplated embodiments and is intended to neither identify critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a composition that can eliminate a wide range of odors from hands.

It is another objective of the present invention that the composition can be used in a variety of hygiene products.

It is still another object of the present invention that the composition can be used in a handwash formulation.

It is yet another object of the present invention that the composition can be used to eliminate odors that are hard to remove.

In one aspect, disclosed is a synergetic composition that includes a combination of Soya based Onium compounds and Sodium Alginate. The disclosed composition can eliminate a wide range of odors from the hands.

In one aspect, the composition can be used in a variety of formulations, such as handwash, wipes, spray cleaners, floor cleaners, and laundry products.

### BRIEF DESCRIPTION OF THE DRAWINGS

The accompanying FIGURES, which are incorporated herein, form part of the specification and illustrate embodiments of the present invention. Together with the description, the FIGURES further explain the principles of the present invention and enable a person skilled in the relevant arts to make and use the invention.

FIGURE is a chart showing the synergetic effect of the combination of soya-ethyl morpholinium ethosulfate (SME) and sodium alginate (ALG), according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, the reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is to describe particular embodiments only and is not intended to be limiting to embodiments of the invention. As used herein, the singular forms “a”, “an”, and “the” are intended to include the plural forms as well, unless the context indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention since the scope of the invention will be best defined by the allowed claims of any resulting patent.

A novel composition is disclosed that can eliminate a wide range of odors from the skin including that of the hands. The disclosed composition can eliminate hard-to-remove odors, such as garlic and formaldehyde. The disclosed composition can be used in a variety of hand hygiene formulations, such as handwashes, wipes, spray cleaners, and the like. Besides hand hygiene, the disclosed composition can also be used for various bodily malodors. The disclosed composition can also be used in floor cleaners, laundry products, and the like to eliminate malodors.

The disclosed composition can include ingredients that can synergistically act to eliminate malodors. The disclosed composition can include Soya based Onium compounds and Sodium Alginate as the chief ingredients. The soya-based onium compounds include soya-ethyl morpholinium ethosulfate (SME). Both components are essential ingredients that result in a synergetic effect in eliminating odors from the skin. The SME is a highly effective odor eliminator that reacts with sulfur and amine malodors, however, the use of SME has certain limitations, such as residual odor in many cases. The use of Sodium Alginate in combination results in a synergetic effect providing a better effect than either ingredient can achieve, by itself. Sodium Alginate can act as an adsorber which helps scavenge residual odor. The synergistic effect was observed having the SME:ALG proportions in a range of about 90:10 to 97:3 wt (%), wherein the optimum proportion of the SME and ALG in the hygiene compositions can be about 95:5.

Referring to FIGURE which is a chart showing the effectiveness of the disclosed combination of SME and sodium alginate for different odors. In the case of body odor, the combination of SME and sodium alginate provided better odor control eliminating any residual odor. This residual odor could not be eliminated by either SME or ALG alone. In the case of smoke, a significant improvement in odor control was observed. Eliminating the smoke odor has been a challenge, however, the disclosed combination can effectively control smoke-related odor. Significant synergistic effects were also observed in bathroom-related odors, wherein either the SME or sodium alginate was ineffective alone.

### Example 1: Handwash Formulation

| Ingredients | Wt. (%) |
|---|---|
| SME | 1 |
| Sodium alginate | 0.05 |
| Sodium Laureth | 12 |
| Sulfate | |
| Glycerine | 0.25 |
| Aloe (200×) | .05 |
| EDTA | 1 |
| Phenoxyethanol | 0.5 |
| Citric Acid | 0.25 |
| NaCl | 1 |
| Xanthan Gum | 0.1 |
| Fragrance | 0.75 |
| Water | Q.S. |

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. An odor control composition consisting essentially of:
soya-ethyl morpholinium ethosulfate (SME) and
sodium alginate (ALG), wherein SME:ALG is about 90:7 to 97:3.

2. The odor control composition according to claim 1, wherein SME:ALG is about 95:5 to 97:3.

3. A handwash formulation comprising:
soya-ethyl morpholinium ethosulfate; and
sodium alginate.

4. A method of eliminating odor, the method comprising:
providing a formulation comprising:
soya-ethyl morpholinium ethosulfate, and
sodium alginate; and
applying the formulation to an area having odor.

5. The method according to claim 4, wherein the odor is smoke odor.

6. The method according to claim 4, wherein the odor is body odor, and the formulation is a handwash.

7. The method according to claim 4, wherein the odor is bathroom odor, and the formulation is applied to air.
